(19) United States
(12) Patent Application Publication
(10) Pub. No.: US 2012/0141587 A1
(43) Pub. Date: Jun. 7, 2012

(54) COMPOSITIONS AND METHODS FOR TREATMENT OR PREVENTION OF POST-OPERATIVE ORGAN OR TISSUE INFLAMMATION

(76) Inventors: Dale Yoo, Atlanta, GA (US); Sean Parick Whalen, Nashville, TN (US); Jonathan Langberg, Atlanta, GA (US)

(21) Appl. No.: 13/382,771

(22) PCT Filed: Aug. 25, 2010
(86) PCT No.: PCT/US10/46621

§ 371 Date: Jan. 6, 2012

Related U.S. Application Data
(60) Provisional application No. 61/236,726, filed on Aug. 25, 2009.

(51) Int. Cl. A61K 31/58; A61K 9/00; A61P 29/00

(52) U.S. Cl. 424/484; 514/174

(57) ABSTRACT

Compositions, methods and kits for treatment or prevention of post operative organ or tissue inflammation are provided. The compositions contain an effective amount for local delivery, of an anti-inflammatory agent, alone or in combination with other pharmacologic agents, embedded within a polymeric matrix or gel. The polymeric matrix or gel may be a formed from natural or synthetic precursor components. The compositions are applied locally to an organ or tissue for the treatment or prevention of post inflammation resulting from surgical intervention.

### FIELD OF THE INVENTION

The present invention is related to the preparation and use of polymeric matrices which incorporate anti-inflammatory agents, for local treatment or prevention of post operative organ or tissue inflammation.

### BACKGROUND OF THE INVENTION

Cardiac arrhythmias, such as atrial fibrillation, are a commonly occurring disorder characterized by erratic beating of the heart following procedures such cardiac or pulmonary surgery. Post operative atrial fibrillation (POAF) occurs in up to 40% of patients following open heart surgery, and remains a common cause of morbidity and prolonged hospital stay (Maisel, et al., Ann. Inter. Med., 135:1061-1073 (2001). Atrial fibrillation is characterized by rapid randomized contractions of atrial myocardium, causing an irregular, often rapid ventricular rate. The regular pumping function of the atria is replaced by a disorganized, ineffective quivering as a result of chaotic conduction of electrical signals through the upper chambers of the heart. Atrial fibrillation is often associated with other forms of cardiovascular disease, including congestive heart failure, rheumatic heart disease, coronary artery disease, left ventricular hypertrophy, cardiomyopathy, or hypertension.

Post operative atrial fibrillation is a significant problem for hospitals worldwide with no effective solution. It is the most common morbidity event after coronary bypass grafting. Atrial arrhythmias occur after cardiac surgery in 10% to 65% of patients (Aranki, et al., Circulation, 94:390-7 (1996); Mathew, et al. JAMA, 276:300-6 (1996); Zaman, et al., Circulation, 101:1403-8 (2000) and William, et al., Ann. Int. Med. 135(12):1063-1073 (2001)). It has been estimated that the incidence of atrial fibrillation following coronary artery bypass graft (CABG) surgery is between 25% and 40%. The rate is even higher for patients undergoing valve surgery either alone or in combination with CABG surgery. Although the atrial fibrillation may resolve itself within the first ten days following surgery, the problem is associated with high levels of morbidity during the post operative phase and can increase the cost of hospital stays by $20,000 or more.

Spontaneous conversion of atrial fibrillation after cardiac surgery is common; 15% to 30% of patients convert within 2 hours (Gavaghan, et al., Aust N Z J Med, 15:27-32 (1985) and VanderLugt, Circulation, 1999;100:369-75 (1999)) and 25% to 80% of patients convert within 24 hours when either digoxin alone or no antiarrhythmic therapy is administered (Campbell, Aust N Z J Med., 10:644-9 (1980) and Cochrane, et al., Eur J Cardiothorac Surg., 8:194-8 (1994)). Two management strategies are available to treat patients with recurrent atrial fibrillation: cardioversion and rate control. Cardioversion is the conversion of the abnormal atrial rhythm into normal sinus rhythm. This conversion is generally achieved pharmacologically or electrically. Rate control therapy is used to control the ventricular rate, while allowing the atria to continue fibrillation. This is generally achieved by slowing the conduction of signals through the AV node from the atria to the ventricles. Thus, in response to postoperative atrial fibrillation, current practice dictates pharmacological treatments and/or electrical shock conversion. However, maintaining the sinus rhythm after successful cadioversion has proven to be quite difficult and, therefore, the recurrent rate is extremely high without systemic administration (orally or intravenously) of anti-arrhythmic drugs. For this reason, it is mandatory to administer anti-arrhythmic drugs for at least the recovery period. Such systemic administration of drugs can be associated with significant side effects.

The pathogenesis of atrial fibrillation is unknown, but studies have supported a role for both oxidative stress and inflammation. Studies of animal and human samples have shown increased myocardial oxidative stress associated with atrial fibrillation (Cai, et al., Circulation, 106:2854-2858 (2002); Dudley, et al., Circulation, 112:1266-1273 (2005); Kim, et al., Circ Res., 97:629-636 (2005); and Mihm, et al., Circulation, 104:174-180 (2001)). Anti-oxidants including statins, Vitamin C, polyunsaturated fatty acids, and fish oils have been shown to reduce the incidence of atrial fibrillation in humans (Amar, et al., Chest, 128:3421-3427 (2005); Calo, et al., J Am Coll Cardia, 45:1723-1728 (2005); Carnes, et al., Circ Res., 89:E32-E38 (2001); Korantzopoulos, et al., Int J Cardiol., 102:321-326 (2005); and Korantzopoulos, et al., Int J Clin Pract., 59:881-885 (2005)).

Inflammation has a complex relationship with oxidative stress and has also been associated with atrial fibrillation. There is evidence of inflammatory infiltrates in many cases of lone atrial fibrillation (Frustaci, et al., Circulation, 96:1180-1184 (1997). Elevated levels of the inflammatory marker, C-reactive protein (CRP), are associated with atrial fibrillation in some studies (Aviles, Circulation, 108:3006-3010 (2003); Chung, et al., Circulation, 104:2886-2891 (2001); and Dernellis, et al., Acta Cardiol., 56:375-380 (2001)), and have been suggested as a predictor of the incidence of atrial fibrillation following cardioversion (Dernellis, et al., Acta Cardiol., 56:375-380 (2001); Korantzopoulos, et al., Int J Clin Pract., 59:881-885 (2005); Malouf, et al., J Am Coll Cardiol., 46:1284-1287 (2005) and Watanabe, et al., Int J Cardiol., 108:346-353 (2006)), or cardiac surgery (Fontes, et al., Anesth Analg. 101:17-23 (2005) and Lo, et al., Ann Thorac Surg. 79:1530-1535 (2005)). Interleukin-6 (IL-6) is elevated in atrial fibrillation (Conway, et al., J Am Coll Cardiol., 43:2075-2082 (2004)), and polymorphisms in the IL-6 gene predict the onset of atrial fibrillation after cardiac surgery (Gaudino, et al., Circulation, 108(Suppl 1):II195-II199 (2003)). Anti-inflammatory agents may reduce the risk of atrial fibrillation (Dernellis, et al., Eur Heart J., 25:1100-1107 (2004); Dernellis, et al., Am Heart J., 150:1064 (2005); Ishii, et al., Circulation, 111:2881-2888 (2005); Mathew, et al., JAMA, 291:1720-1729 (2004); Prasongsukarn, et al., J Thorac Cardiovasc Surg., 130:93-98 (2005); Shiroshita-Takeshita, et al., Cardiovasc Res., 69:865-875 (2006) and Yared, et al., Ann Thorac Surg., 69:1420-1424 (2000)). For example, oral prednisone was shown to prevent inducible atrial flutter in a canine model of sterile pericarditis model (Goldstein, et al., J. Cardiaocas. Electrophys. 19(1):74-81 (2007).

It is desirable to deliver drugs to the atrial tissues without administering the drugs systemically. Such delivery directly to the atrial tissue should require only a fraction of the systemic dose of the drugs to adequately raise their concentration in the atrial tissue, thereby achieving anti-atrial fibrillation effects without inducing systemic side effects, which are common for the systemic administration of anti-inflammatory agents, particularly steroidal anti-inflammatory agents.

Drugs have been delivered directly to the pericardial sac after transvenously passing a catheter to the right atrium and thereafter transatrially puncturing the atrial wall, thereby providing access to the pericardial sac. See, e.g., U.S. Pat. Nos. 5,269,326 and 5,968,010. Maisch, et al., Eur. Heart J., 23(19): 1503-1508 (2002), show intrapericardial delivery of traimcinolone for treatment of autoreactive pericardial effusion. However, direct administration requires removal of the drug solution either by implanting a shunt to drain the drug solution from the pericardial sac or by physically removing the drug solution via a syringe or other device. Both of these methods involve additional trauma to the heart and the potential for infection due to repeated opening of the chest cavity. Other approaches include epicardial drug delivery, which can increase efficacy and reduce systemic side effects, but is impractical for POAF because postoperative pericardial effusion/drainage causes rapid drug elimination.

It is therefore an object of the present invention to provide compositions for the treatment of post operative organ inflammation with increased contact time with the site of treatment.

It is a further object of the invention to provide kits and methods for treating post operative organ inflammation.

### SUMMARY OF THE INVENTION

Provided are compositions for treatment of post operative organ inflammation which provide increased contact time with the site of treatment. The compositions contain an effective amount for local delivery, of an anti-inflammatory agent, dispersed within a polymeric matrix or gel. The polymeric matrix may be a formed from natural precursor components or from synthetic precursor components. In a preferred embodiment, the matrix or gel is formed from natural precursor components. In a more preferred embodiment the matrix is a fibrin matrix. Most preferred are hemostatic agents such as fibrin sealants. The composition may optionally contain, in addition to the anti-inflammatory agent, other pharmacologic agents commonly used in locally delivered anti-inflammatory formulations. The compositions are used for local delivery of an effective amount of one or more anti-inflammatory pharmacologic agents, for the treatment of post operative inflammation. In one embodiment, the composition is used for epicardial delivery of the pharmacologic agent. In this embodiment, the anti-inflammatory is a glucocorticoid, preferably triamcinolone.

Also provided is a method for treating post operative inflammation in an organ, by local application of a polymeric matrix or gel containing an effective amount for local delivery, of an anti-inflammatory agent. In one embodiment, the polymeric matrix or gel is applied to the epicardium for the prevention or treatment of post operative atrial fibrillation. Hemostatic agents sprayed on the epicardium vigorously adhere to the epicardium, allowing an admixed drug to remain in contact with the heart despite the presence of an effusion.

Also provided is a kit for the treatment or prevention of post operative inflammation in organs, containing a pharmacologic agent, one or more precursor components that are capable of forming a three-dimensional matrix, clot or gel when combined or polymerized, and instructions for combining the different components of the kit. In some embodiments, the kit also contains a calcium source. The kits may also contain as one or more devices for mixing and/or applying the components such as syringes, pipettes, pipette bulbs, vials, and the like.

### DETAILED DESCRIPTION OF THE INVENTION

Definitions

The term “fibrinogen” includes not only fibrinogen per se, but also any clotting forming substance, such as clot-forming derivatives of fibrinogen.

As generally used herein, the term “thrombin” includes thrombin per se, as well as any gelation-inducing or clot-inducing agent for fibrinogen or fibrin.

“In situ formation” as generally used herein refers to the ability of mixtures of precursor components which are substantially not crosslinked prior to injection to form covalent linkages with each other under physiological conditions (e.g., temperature, pH, etc.) during application and at the site of application in the body of the animal host.

“Cross-linking” as generally used herein means the formation of covalent linkages.

The terms “prevent”, “preventing” and “prevention” as generally used herein refer to inhibiting completely or partially a biological response, as well as inhibiting an increase in a biological response.

“Matrix” as generally used herein refers to a material intended to interface with biological materials, such as tissues, organ, etc. The matrix serves as a delivery device for the bioactive agent incorporated therein. The matrices described herein are formed from liquid precursor components which are able to form a three dimensional network at the desired site. The terms “matrix” or “gel” are used synonymously herein. The terms “matrix” and “gel” refer to the composition formed after the precursor components are mixed together. Thus, the terms “matrix” and “gel” encompass partially or fully crosslinked polymeric or non-polymeric networks. The matrix may be in the form of a liquid, semi-solid, such as a paste, or a solid. Depending on the type of precursor materials, the matrix may be swollen with water but not dissolved in water, i.e. form a hydrogel which stays in the body for a certain period of time.

“Fibrin matrix” as generally used herein means the product of a process in which substantially all of the precursor components, fibrinogen and thrombin, crosslink in the presence of a calcium source to form a three-dimensional network.

“Synthetic precursor molecules” as generally used herein refers to molecules which do not exist in nature.

### I. Adhesive Compositions Containing Anti-Inflammatory Drugs

A. Matrix Materials

The matrix is formed by crosslinking ionically, covalently, or by combinations thereof, one or more polymeric or non-polymeric materials to form a matrix. The crosslinked matrix may form a gel. A gel is a material in which a crosslinked polymer network is swollen to a finite extent by a continuous phase of an aqueous solution. The precursor components may be monomers, oligomers, polymers, and/or small molecule crosslinking agents.

In the preferred embodiment, the matrix is formed of proteins, most preferably proteins naturally present in the patient into which the matrix is to be implanted. The most preferred protein is fibrin, although other proteins such as collagen and gelatin can also be used. Alternatively, the matrix may be formed of polysaccharides and glycoproteins. In some embodiments, it is also possible to use synthetic polymers formed from synthetic precursors, which are crosslinkable by ionic or covalent binding to form the matrix. The matrix material is preferably biodegradable by naturally present enzymes and/or by hydrolysis.

Synthetic Matrices

Synthetic polymeric matrices for application to the human or animal body can be prepared in a variety of ways. Some biomaterials are prepared through free-radical polymerization between two or more precursor components containing unsaturated double bonds, such as described in Hem, et al., J. Biomed. Mater. Res., 39:266-276 (1998). Other biomaterials are prepared by reacting a first precursor component containing two or more nucleophilic groups, X, with at least a second precursor component containing two or more electrophilic groups, Y, which are capable of cross-linking with the nucleophilic group on the first precursor component. The reaction mechanism involved can be a nucleophilic substitution reaction, such as disclosed in U.S. Pat. No. 5,874,500, a condensation reaction and/or a Michael type addition reaction, such as described in WO 00/044808. A detailed description of synthetic polymeric matrices is provided for example in U.S. Published Publication Nos. 2006/0147443 and 2007/0010440, by Schense, et al. and U.S. Pat. No. 7,413,739 to Hubbell, et al.

Fibrin Matrices

Fibrin is a natural material which has been reported for several biomedical applications. Fibrin has been described as a material for cell in-growth matrices in U.S. Pat. No. 6,331,422 to Hubbell et al. Fibrin gels have been used as sealants because of their ability to bind to many tissues and its natural role in wound healing. Some specific applications include use as a sealant for vascular graft attachment, heart valve attachment, bone positioning in fractures and tendon repair. Additionally, these gels have been used as drug delivery devices, and for neuronal regeneration. Although fibrin provides a solid support for tissue regeneration and cell in-growth, there are few active sequences in the monomer that directly enhance these processes.

The process by which fibrinogen is polymerized into fibrin has also been characterized. Initially, a protease cleaves the dimeric fibrinogen molecule at the two symmetric sites. There are several possible proteases than can cleave fibrinogen, including thrombin, reptilase, and protease III, and each one severs the protein at a different site. Once the fibrinogen is cleaved, a self-polymerization step occurs in which the fibrinogen monomers come together and form a non-covalently crosslinked polymer gel. This self-assembly happens because binding sites become exposed after protease cleavage occurs. Once they are exposed, these binding sites in the centre of the molecule can bind to other sites on the fibrinogen chains, which are present at the ends of the peptide chains. In this manner, a polymer network is formed. Factor XIIIa, a transglutaminase activated from Factor XIII by thrombin proteolysis, may then covalently crosslink the polymer network. Other transglutaminases exist and may also be involved in covalent crosslinking and grafting to the fibrin network.

Once a crosslinked fibrin gel is formed, the subsequent degradation is tightly controlled. One of the key molecules in controlling the degradation of fibrin is 2-plasmin inhibitor. This molecule acts by crosslinking to the chain of fibrin through the action of Factor XIIIa. By attaching itself to the gel, a high concentration of inhibitor can be localized to the gel. The inhibitor then acts by preventing the binding of plasminogen to fibrin and inactivating plasmin.

A fibrin matrix is preferably formed from two precursor components which can be in the form of solutions, dry powders, or combinations thereof. The first precursor component, typically in form of a solution, contains fibrinogen, the second precursor component, also typically in form of a solution, contains thrombin. Additionally a calcium ion source is required. The fibrin matrix can be formed using methods known to one of ordinary skill in the art (See U.S. Pat. Nos. 7,229,959, and 5,219,328), or acquired from commercial suppliers of fibrin sealants. For example, TISSEEL® (fibrin sealant) is supplied by Baxter, Healthcare Corp., Deerfield, Ill.

1. Fibrinogen

The first precursor solution contains fibrinogen, preferably in a concentration range between 10 to 130 mg fibrinogen per milliliter of precursor solution, more preferably between 30 to 120 mg fibrinogen per milliliter of precursor solution, even more preferably from between 40 to 110 mg fibrinogen per milliliter of precursor solution, and most preferably 50 mg fibrinogen per milliliter of precursor solution. Fibrinogen is preferably solubilized in an aqueous buffer solution. Even more preferably, the fibrinogen dilution buffer comprises water, sodium citrate, preferably at a concentration of 25 mM, niacinamide, preferably at a concentration of 50 mM and histidine, preferably at a concentration of 100 mM, and has a preferably a pH of 7.3.

2. Thrombin

In a preferred embodiment, the second precursor solution contains thrombin, which is preferably solubilized in an aqueous buffer solution.

The concentrations of the fibrinogen solution and/or the thrombin solutions have a significant effect on the density of the formed network and on the clotting or crosslinking speed of the final fibrin matrix or foam. Typically, the reduction of the amount of thrombin slows down the crosslinking process and contributes to form fibrin matrices with a less dense network. By varying the ratio of thrombin to fibrinogen, one can vary the release rate of the bioactive active.

3. Calcium Source

A calcium ion source may be present in at least one of the precursor solutions or preferably in the bioactive agent solution. The calcium ion source is preferably CaCl₂.2H₂O, preferably in a concentration range between 1 to 10 mg per ml of precursor solution, even more preferable between 4 to 7 mg per ml of precursor solution, most preferably between 5 to 6 mg per ml of precursor solution.

B. Pharmacologic Agents
Anti-inflammatory drugs
1. Steroidal anti-inflammatory drugs

In some embodiments, the pharmacologic agent is a steroidal antiinflamatory agent. Suitable steroidal anti-inflammatory agents include, but are not limited to, triamcinolone acetonide, methylprednisone, triamcinolone acetate, betamethasone, cortisol, desoximetasone, mometasone furoate, fluocinolone acetonide, clocortolone privalate, fluticasone propionate, hydrocortisone butyrate, predincarbate, aclometasone dipropionate, desonide, hydrocortisone probutate, cortisone, fludrocortisone, prednisolone, and dexamethasone. The dose of the steroidal anti-inflammatory agent can readily be determined by the attending physician, dependent on a variety of factors including the organ being treated. In one embodiment, the concentration of the drug is from 1% to about 90% by weight of the composition. Preferably, form about 1% to about 60%, more preferably from about 1% to about 40%, even more preferably from about 1% to about 30%, most preferably from about 1% to about 20%. The steroidal anti-inflammatory agents can be used alone or in combination and may be administered as the free acid or free base or a pharmaceutically acceptable salt thereof. Alternatively, the steroidal anti-inflammatory agent(s) can be co-administered with one or more non-steroidal anti-inflammatory agents.
2. Non-steroidal anti-inflammatory drugs

Non-steroidal anti-inflammatory drugs (NSAIDs) alleviate pain by counteracting the cyclooxygenase (COX) enzyme. On its own COX enzyme synthesizes prostaglandins, creating inflammation. In whole the NSAIDs prevent the prostaglandins from ever being synthesized, reducing or eliminating the pain. These include but are not limited to salicylic acid derivatives such as aspirin, sodium salicylate, olsalazine, para-aminopenenol derivatives such as acetaminophen, heteroacyl acetic acids such as tomeltin, diclofenac or ketoralac, or arylpropionic acids such as ibuporfen, naproxen, fluriprofen, ketoprofen, fenoprofen and oxaprozin.
3. Other Pharmacologic agents

The formulations described herein may optionally contain one or more other pharmaceutically active agents. Useful agents include any agents commonly used in cardiac formulations or in anti-inflammatory formulations

Among the clinically significant pharmacologic agents (i.e., drugs) which could advantageously be delivered to the heart via in the formulations of the present invention are those which improve cardiac contractility (e.g., digitalis drugs, adrenergic agonists, etc.), suppress arrhythmias (e.g., class I, II, III, and IV agents and specialized drugs such as amiodarone, which is particularly potent but has severe systemic side effects), dilate coronary arteries (e.g., nitroglycerin, calcium channel blockers, etc.), and lyse clots in the coronary circulation (e.g., thrombolytic agents such as streptokinase or tissue-type plasminogen activator (TPA)). Procainamide is well known to infiltrate tissue readily and amiodarone is a lipophylic substance and is expected to infiltrate atrial tissue as well as procainamide. Ayers et al. demonstrated the effectiveness in suppressing atrial fibrillation using amiodarone instilled into a canine's pericardial sac (Ayers, et al., J Cardiovasc Electrophysiol., 7(8):713-21 (1996). Other antiarythmic agents include quinidine, disopyramide, lidocaine, phenytoin, mexiletine, ibutilide, sotalol, defetilide, propafenone, and flecainide. Other drugs include digoxin, metoprolol, esmolol and verapamil.

Additional agents which can be included in the polymeric matrix or gel antibiotics; antiproliferative/cytotoxic drugs; antivirals; cytokines; colony stimulating factors; erythropoietin; antifungals; antiparasitic agents; steroids; anesthetics; analgesics; oncology agents; and hormones.

Other compounds include, but are not limited to: vitamins and other nutritional supplements; hormones; peptides and proteins; carbohydrates; and gene therapy reagents. Genetically altered cells, stem cells, and/or other cells may also be included in the composition. For some applications, cell growth factors may also be added to the composition to promote rehabilitation of damaged tissue and/or growth of new, healthy tissue.

### II. Kits

In one embodiment, the kit for the treatment or prevention of post operative inflammation in organs, contains one or more pharmacologic agents and one or more precursor components capable of forming a three-dimensional matrix, clot or gel when combined or polymerized. In some embodiments, the kit also contains an initiator to initiate reaction of the precursor components to form the matrix. In one embodiment, the initiator is a calcium source. The kits may also contain one or more devices for mixing and/or applying the components such as syringes, pipettes, pipette bulbs, vials, and the like. The components of the kit are all housed in an appropriate container or package, such as a cardboard box.

The precursor components (may be in the form of a solid, such as a dry powder or may in solution, such as in a buffer. If present, the calcium source may be in the form of a powder or in solution. If the precursor components are in the form of a solid, the kit may contain buffer solutions and instructions for preparing solutions of the precursor components.

In one embodiment, the precursor components in the kit are provided in a two-way syringe device. The components, active agent(s), carriers, excipients, etc. are mixed by squeezing the contents of both syringes through a mixing chamber and/or needle and/or static mixer.

In a preferred embodiment, the first component (a) in the kit is fibrinogen and the second precursor component (b) is thrombin which, when combined together, form a fibrin matrix. Fibrinogen is dissolved (optionally with aprotinin to increase stability) in a buffer solution at physiological pH (in a range from pH 6.5 to 8.0, preferably from pH 7.0 to 7.5) and is stored separately from a solution of thrombin, optionally in a calcium chloride buffer (e.g. concentration range of from 40 to 50 mM). The buffer solution for the fibrinogen can be a histidine buffer solution at a preferred concentration of 50 rnM including additionally NaCl at a preferred concentration of 150 mM or TRIS buffer saline (preferably at a concentration of 33 mM).

Preferably both fibrinogen and thrombin are stored separately from each other in lyophilized form. Prior to use, a tris or histidine buffer is added to the fibrinogen, and the thrombin is dissolved in a calcium chloride solution. Subsequently, the fibrinogen and the thrombin solutions are placed in separate containers/vials/syringe bodies and mixed by a two way connecting device, such as a two-way syringe. Optionally, the containers/vials/syringe bodies are bipartite devices, having two chambers separated by an adjustable partition which is perpendicular to the syringe body wall. One of the chambers contains the lyophilized fibrinogen or thrombin, while the other chamber contains an appropriate buffer solution. When the plunger is pressed down, the partition moves and releases the buffer into the fibrinogen chamber to dissolve the fibrinogen. Once both fibrinogen and thrombin are dissolved, both bipartite syringe bodies are attached to a two-way connecting device and the contents are mixed by squeezing them through the injection needle attached to the connecting device. Optionally, the connecting device contains a static mixer to improve mixing of the contents.

In one embodiment an active agent triamcinolone acetate was purchased as a kit, sold under the trade name KENALOG™ manufactured by Westwood-Squibb Pharmaceutical, Inc. (New York, N.Y.). Each vial in the kit contains 40 mg triamcinolone acetonide in 1 mL Nacl. It is within the abilities of an attending physician to determine how many vials would be effect to treat or prevent POAF in an organ of choice.

### III. Methods of Use to Treat or Reduce Post-Surgical Inflammation

A. Methods of Preparing a Polymeric Matrix

The polymeric matrix or gel is prepared by mixing the precursor components, initiating polymerization, or combinations thereof. Preferably a homogenous mixture is formed. Synthetic precursor components and methods for preparing synthetic polymeric materials are described in detail in U.S. Pat. No. 7,413,739 to Hubbell, the contents of which are herein incorporated by reference.

For fibrin matrices, a fibrinogen solution can be transferred into a 10 cc luer-lock syringe, and the thrombin solution can be transferred into a 10 cc leer-lock syringe.

The fibrinogen solution can be prepared in an appropriate buffer, for example histidine buffer solution at a preferred concentration of 50 mM including additionally NaCl at a preferred concentration of 150 mM or TRIS buffer saline (preferably at a concentration of 33 mM). The solution is homogenized and centrifuged to remove bubbles and sterilized, such as by filtering through a 0.22 μm filter. The thrombin solution can be prepared in a thrombin dilution buffer, for example a buffer containing about 40 mM CaCl₂ in double-distilled water. The solution is homogenized and centrifuged to remove bubbles and sterilized, such as by filtering through a 0.22 μm filter.

In the preferred embodiment, the matrix material is gelled in situ in or on the organ being treated. To prevent premature contact prior to administration, a kit which separates the precursor solutions from each other may be used. Upon mixing under conditions that allow polymerization, the compositions form a bioactive factor supplemented three-dimensional network. Depending on the precursor components and their concentrations, gelling can occur quasi-instantaneously after mixing. In one embodiment, the gelling time is less than 3 minutes, preferably less than two minutes, preferably less than one minute, more preferably less than 30 seconds, and most preferably less than 10 seconds. The solutions are preferably mixed by a two way syringe device, in which mixing occurs by squeezing the contents of both syringes through a mixing chamber and/or needle and/or static mixer.

In some embodiments, the syringes containing the first precursor component and the second precursor component are connected through a luer lock adapter and their content is homogenized by transferring the contents from syringe to syringe thoroughly. The mixture can then be sprayed onto a target tissue. The preparation of the matrix composition can be carried out at any suitable temperature, such as in the range from about 18 to about 37° C., for example 25° C. In order to suspend or disperse the pharmacologic agents within the resulting matrix, the precursor components are reconstituted in the presence of pharmacologic agents, thus ensuring that treatment agents are incorporated into the resulting polymeric matrix composition and the matrix becomes a vehicle for the delivery of these compounds to the organs to be treated. In addition, compounds which stabilize or extend the longevity of the fibrin sealant may be added to the mixture. Generally, these compounds are poorly soluble in water. Therefore, such compounds may increase the duration of drug, or similar agent, release from the fibrin matrix and enhance the ability of the sealant to deliver localized dosages.

In any of the embodiments described herein, the bioactive agent may be added to one or more of the precursor components before they are mixed to form the polymeric matrix. Alternatively, the bioactive agent can be added to the components as they are being mixed to form the matrix.

In a preferred embodiment the matrix is formed from fibrinogen. Fibrinogen, through a cascade of various reactions gels to form a matrix, when brought in contact with thrombin and a calcium source at appropriate temperature and pH. An exogenous pharmacologic agent, such as triamcinolone acetonide, is incorporated within the fibrin during coagulation. In some embodiments, more than one pharmacologic agent is incorporated into the matrix. The pharmacologic agent is embedded within a fibrin matrix for example by providing a fibrinogen or thrombin solution containing the pharmacologic agent. For freeze-dried precursor components, reconstitution of the thrombin, for example, can be accomplished using a solution containing calcium chloride in combination with the pharmacologic agent. Preferably, the thrombin reconstituting solution, with the desired pharmacologic agent(s), is prepared in a single vial prior to mixing with the freeze-dried thrombin. This component may then be provided to users in a reconstituted state, or in two uncombined vials containing freeze-dried thrombin and a premixed reconstitution solution. Mixing of the contents of the two vials may be performed at any point up to, and including, the time at which the precursor mixture is sprayed onto a target organ.

Alternatively, a solution containing the pharmacologic agent may be used to reconstitute fibrinogen. For example the pharmacologic agent may be provided in the aprotinin solution, or in the buffer for reconstituting fibrinogen.

In some embodiments the final mixing of the precursor components occurs in a needle mounted on a Y-connector which connects a dual syringe system. This method of preparation facilitates the formation of the matrix at the desired site during delivery, or immediately thereafter. The pharmacologic agent(s) chosen depend upon the indications of the particular patient and the specific application. For example, a corticosteroid-containing solution such as, for example, a triamcinolone solution is used to reconstitute the thrombin from its freeze-dried state for the treatment or prevention of POAF. Freeze-dried fibrinogen is reconstituted according to conventional means and the individual precursor components are loaded into the separate receivers of the Y-connector for subsequent injection. In some embodiments the bioactive agent is added to freeze-dried preparations; however, the bioactive agent may also be added to fresh fibrinogen or thrombin or added to thawed/frozen. Freeze-dried or fibrinogen and freeze-dried or frozenbin are available in kit-form from manufacturers such as Baxter under names such as TISSEEL®.

The volume of the precursor solutions used will be dependent on the on the surface area that needs to be covered.

B. Methods of Using the Polymeric Matrix

The formulation containing the pharmacologic agent can be used to treat any organ exhibiting post surgical inflammation. The dosage of the pharmacologic agent(s) will be dependent on the condition being treated, and can be readily deteunined by one of ordinary skill in the art. Conditions to be treated for example are inflammation resulting from cardiac patients undergoing cardiopulmonary bypass; vascular prosthesis implantation; coronary artery bypass grafting; lung surgery; organ transplantation, including but not limited to heart, renal, liver, lungs, pancreas, penis, and intestine transplantation; or tissue transplantation, included but not limited to bones, tendons, cornea, heart valves, veins, extremities, such as hands, feet, arms, and legs; skin transplantation; and vascular surgery. In a preferred embodiment, the compositions are used to prevent and/or treat post-operative inflammation in internal organs, such as the heart, lungs, kidneys, liver, etc.

The scope of the invention can be understood by the following non-limiting example.

### EXAMPLES

Materials

Talc (5 gm sterile talc) was obtained from Bryan Corporation (Woburn, Mass.), and calcium chloride was obtained from Sigma-Aldrich (St. Louis, Mo.).

TISSEEL® (fibrin sealant) was obtained from Baxter, Healthcare Corp., Deerfield, Ill. KENALOG™ (triamcinolone acetonide) (1 ml vials, 40 mg triamcinolone per 1 ml) is manufactured by Westwood-Squibb Pharmaceutical, Inc. (New York, N.Y.).

Methods

Talc was instilled into the pericardium in fifteen crossbred hounds to simulate postoperative inflammation as previously described (Page, et al., J Am Coll Cardiol, 8:872-879 (1986). The pericardium was opened directly above the left atrium and the left ventricle using scissors, after the “talc bath”. Permanent pacemakers were implanted to monitor atrial arrhythmias. An algorithm built into the MEDTRONIC INSYNC® device (pace maker) which counts total seconds/minutes in atrial fibrillation based on rate, dyssynchrony between the atrial lead and ventricular lead as well as variability and onset was used to measure POAF.

A solution containing triamcinolone and calcium chloride which was used to reconstitute thrombin was made by dissolving calcium chloride powder into a 1 ml vial of KENALOG™ (triamcinolone acetonide). Briefly, 5.88 mg of CaCl₂.2H₂O was mixed with the KENALOG™ solution (40 mg triamcinolone acetonide in 1 ml in NaCl) to make a 40 μmol/mL solution. This solution was then added to the 1 thrombin vial. 2 TISSEEL® (fibrin sealant) kits were used per dog. Each 2 mL kit contained 1 mL thrombin and 1 mL fibrinogen. The mixture of triamcinolone acetonide (80 mg)+calcium chloride, and fibrin sealant was sprayed onto the atria of the study group animals (n=9), while control animals (n=6) received only TISSEEL® (fibrin sealant). A total volume of 4 mL of fibrinogen/steroid sealant was used to cover the anterior and lateral walls of the epicardium.

After the drug was sprayed on, the chest was closed and pacemaker was implanted. The animals were observed for a week after the surgical procedure. After one week, pacemakers were interrogated for mode switch events and excised hearts were subjected to histologic examination and tensile strength testing. Histology/slides were prepped at time of sack. Left atrial appendage paraffin blocks were made and slides were made of these in order to identify and count inflammatory cells microscopically. Healing was also noted during the week.

Results

POAF occurred in 6 out of 6 (100%) of the control animals but only on 3 out of 9 (33%) study group animals (p<0.05). The time in atrial fibrillation per day was 4961±2236 second in controls and 91±52 seconds in the study group (p=0.0176). Severe inflammation (>100 inflammatory cells/40x field) was present in 6 out of 6 animals in the control, versus 1 out of 9 study group animals (p=0.0002). The tensile strength within the atriotomy was not significantly different between the two groups (control=1.18±0.09 N/suture versus study group=0.95±0.10 N/suture (p=0.14). Plasma triamcinolone levels in the study group (0.2 μg/mL) were much less than those seen in previous studies of intraarticular/intravenuous injections. Thus, epicardial delivery of the active agent result in lower systemic drug levels than even intra-articular injections.

The data demonstrates that a mixture of a steroid such as triamcinolone and fibrinogen sealant, sprayed onto the atria can dramatically reduce the burden of POAF and reduce inflammatory cell infiltration. There was no change in the tensile strength of the atrial myocardium and plasma steroid levels were low.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

1. A formulation for local delivery to an organ or tissue comprising
(i) an anti-inflammatory agent; and
(ii) a composition capable of forming a matrix in vivo
wherein the anti-inflammatory agent is dispersed in the matrix and is in an amount effective to treat or prevent post operative organ inflammation.

2. The formulation of claim 2, wherein the precursor components form a fibrin matrix.

3. The formulation of claim 2, wherein the composition capable of forming a matrix comprises more than one precursor components, and wherein the formulation is provided in a kit in which at least one of the components of the composition capable of forming a matrix is stored separately from the other components of the composition.

4. The formulation of claim 1, wherein the anti-inflammatory agent is a steroidal anti-inflammatory agent.

5. The formulation of claim 4, wherein the steroidal anti-inflammatory agent is selected from the group consisting of triamcinolone acetonide, methylprednisone, triamcinolone acetate, betamethasone, cortisol, desoximetasone, Mometasone furoate, fluocinolone acetonide, mometasone furoate, clocortolone privalate, fluticasone propionate, hydrocortisone butyrate, predincarbate, aclometasone dipropionate, desonide, hydrocortisone probutate, cortisone, fludrocortisone, prednisolone, and dexamethasone.

6. The formulation of claim 1 wherein the matrix is a polymeric matrix.

7. The formulation of claim 5, wherein the steroidal anti-inflammatory is triamcinolone acetonide.

8. A method of locally treating or preventing post operative organ or tissue inflammation, comprising
(i) administering to an organ or tissue in need of treatment a formulation comprising an anti-inflammatory agent; and
(ii) a composition capable of forming a matrix
wherein the anti-inflammatory agent is embedded within the matrix and is in an amount effective to treat or prevent post operative organ inflammation.

9. The method of claim 8, wherein the anti-inflammatory agent is embedded with the matrix during its formation.

10. The method of claim 8, wherein the composition capable is sprayed onto the organ or tissue.

11. The method of claim 8, wherein the organ or tissue is selected from the group consisting of heart, kidney, liver, lungs, pancreas, penis, intestine, bone, tendons, cornea, heart valves, veins, arms, and skin.

12. The method of claim 10, wherein the organ is the heart.

13. The method of claim 12, wherein the composition is sprayed onto the epicardium.

14. The method of claim 8 wherein the anti-inflammatory agent is a steroid selected from the group consisting of triamcinolone acetonide, methylprednisone, triamcinolone acetate, betamethasone, cortisol, desoximetasone, Mometasone furoate, fluocinolone acetonide, mometasone furoate, clocortolone privalate, fluticasone propionate, hydrocortisone butyrate, predincarbate, aclometasone dipropionate, desonide, hydrocortisone probutate, cortisone, fludrocortisone, prednisolone, and dexamethasone.

15. The method of claim 14, wherein the anti-inflammatory agent is triamcinolone acetonide.

16-21. (canceled)
